(19) United States
(12) Patent Application Publication
(10) Pub. No.: US 2020/0046753 A1
(43) Pub. Date: Feb. 13, 2020

(54) Modified pT7/T7 Polymerase System for Sustained shRNA Expression in Cytoplasm and Liposome Transporter Comprising the Same

(71) Applicant: KOREA INSTITUTE OF SCIENCE AND TECHNOLOGY, Seoul (KR)

(72) Inventors: Hyung Jun Ahn, Seoul (KR); Seo Young Kwak, Seoul (KR)

(21) Appl. No.: 16/199,815

(22) Filed: Nov. 26, 2018

(30) Foreign Application Priority Data
Aug. 10, 2018 (KR) 10-2018-0093845

(51) Int. Cl. A61K 31/7105; C12N 15/113; A61K 9/127; A61K 31/221; A61K 48/00; A61P 35/00

(52) U.S. Cl. CPC A61K 31/7105; C12N 15/113; A61K 9/1271; A61P 35/00; A61K 48/0058; A61K 48/0075; A61K 48/0066; A61K 31/221

(57) ABSTRACT

The present invention relates to a composition for nucleus-independent, sustained inhibition of gene expression, comprising an mRNA fragment of T7 RNA polymerase, plasmid DNA for self-amplification of the T7 RNA polymerase, and a DNA fragment encoding a gene expression inhibitor; a transporter of the gene expression inhibitor comprising the composition; and a method of preparing the transporter.

The composition of the present invention and the liposome transporter of the present invention comprising the composition can improve the expression of shRNA in the cytoplasm through self-amplification of nucleus-independent, sustained self-amplification of T7 RNA polymerase, and deliver them in a cancer tissue-specific manner. Therefore, the composition and the liposome transporter of the present invention can be utilized for use in treating chronic diseases that require reduced frequency of administration and long-term inhibition of gene expression.

### TECHNICAL FIELD

The present invention relates to a composition for nucleus-independent, sustained inhibition of gene expression, which contains an mRNA fragment of T7 RNA polymerase, plasmid DNA for self-amplification of the T7 RNA polymerase, and a DNA fragment encoding a gene expression inhibitor; a transporter of the gene expression inhibitor containing the composition; and a method of preparing the transporter.

### BACKGROUND ART

RNA interference (RNAi) refers to a phenomenon in which the decomposition of mRNA of a target gene is selectively induced or the expression of the target gene is inhibited by introducing double-stranded RNA, which consists of sense RNA and antisense RNA with a complementary sequence thereto, to cells, etc. RNAi was first discovered in C. elegans, but it is now observed as a very well-preserved life phenomenon in various kinds of animals, plants, microorganisms (e.g., yeast), insects, etc.

Small interference RNA (siRNA), which is an RNAi-inducing material, refers to a short double-helix RNA strand consisting of about 20 to 30 nucleotides. Once an siRNA is injected into a cell, it targets an mRNA with a complementary nucleotide sequence thereto and thereby inhibits the expression of the corresponding gene. siRNAs have an effect of treating diseases, and are attracting attention as an effective method to control life processes being targeted, due to their easy preparation method and high target selectivity.

Currently, the diseases that can be treated using siRNAs under study include cancer, viral infectious diseases, autoimmune diseases, neurodegenerative diseases, etc., and as a clinical trial, the potential of siRNAs for treatment of senile macular degeneration and respiratory syncytial virus infection was reported (Melnikova I. Nat Rev Drug Discov 2007, 6, 863 to 864). Additionally, it was reported that the siRNA delivery system can be employed in human cancer treatment using a cyclodextrin-based nanoparticle polymer targeting transferrin (Oh YK. et al., Adv Drug Deliver Rev 2009, 61, 850 to 862).

However, gene silencing using synthetic siRNAs has a problem in that they have a very short duration of 2 to 4 days, and this is because siRNAs are easily degraded by various nucleases in the cytoplasm and the siRNA concentration is diluted when cell division occurs.

Due to the short duration, there are problems in that not only is frequent injection of synthetic siRNAs required, but also, the efficiency is too low to perform gene silencing of target proteins with a long half-life due to the short duration, and these limitations slow the development of therapeutic agents using siRNAs.

Conventionally, in an effort to improve the duration of gene silencing, attempts were made to deliver as many siRNAs as possible by increasing the loading efficiency of siRNAs using various kinds of transporters. For example, transporters such as Lipofectin®, Lipofectamine®, Cellfectin®, cationic phospholipid nanoparticles, cationic polymers, or liposome-based transporters have mainly been used. However, transporters using cationic molecules or synthetic polymers had problems in that they have low transport efficiency into cells and have cytotoxicity that may be induced during gene transfer into cells. Additionally, in the case of viral vectors capable of exhibiting a long duration of gene silencing, they had problems in that in vivo stability cannot be guaranteed because of immunological side-effects caused by the immunogenicity of the surface protein of these viral vectors, despite their excellent duration. Above all, due to the risk of introducing an exogenous gene into the patient's genome, the application of gene silencing to the human body using a virus has been limited.

Meanwhile, in an effort to improve the duration of gene silencing, plasmid DNA capable of shRNA expression based on a nucleus-dependent promoter has been developed. RNA polymerase III (Pol III) promoters derived from U6 small nuclear RNA and H1 RNA genes are predominantly used, and the shRNA expression in the nucleus using such a nucleus-dependent promoter provides an effective method for long-term production of shRNAs. However, such a plasmid DNA method relying on cell membrane permeability, where the efficiency is known to be as low as 1%, unlike nuclear membrane permeability, has very low efficiency of gene silencing. In particular, the application of plasmid DNA in vivo is limited, as it has very low transfer efficiency in vivo. Additionally, in the case of highly-concentrated shRNAs expressed in the nucleus, they can be converted by a dicer, etc. to mature siRNAs, which can participate in gene silencing, only when they are released into the cytoplasm. However, these exportin-5 transporters involved in the release of shRNAs into the cytoplasm are problematic because they become saturated by highly-concentrated shRNAs, thereby preventing even the release of microRNAs involved in other cellular functions. The problem of the release of microRNAs into the cytoplasm results in serious toxicity. In addition, although methods for nucleus-dependent shRNA expression using various plasmid DNAs have been known, the in vivo model still has limitations due to short duration.

Under the circumstances, the present inventors have made efforts to develop a composition capable of maintaining sustained shRNA expression in the cytoplasm in a nucleus-independent manner. As a result, they have discovered that a composition containing an mRNA fragment of T7 RNA polymerase, plasmid DNA for self-amplification of the T7 RNA polymerase, and a DNA fragment encoding a gene expression inhibitor; and a transporter including the composition can sustain long-term expression of shRNAs in the cytoplasm, and in particular, that they can inhibit the expression of a target gene in a cancer tissue-specific manner, thereby completing the present invention.

### DISCLOSURE

### Technical Problem

An object of the present invention is to provide a composition for sustained inhibition of gene expression, containing an mRNA fragment of T7 RNA polymerase, plasmid DNA for self-amplification of the T7 RNA polymerase, and a DNA fragment encoding a gene expression inhibitor.

Another object of the present invention is to provide a transporter of a gene expression inhibitor including the composition.

Still another object of the present invention is to provide a method for preparing the transporter.

### Technical Solution

Hereinbelow, exemplary embodiments of the present invention will be described in detail. Meanwhile, each of the explanations and exemplary embodiments disclosed herein can be applied to other explanations and exemplary embodiments. That is, all of the combinations of various factors disclosed herein belong to the scope of the present invention. Furthermore, the scope of the present invention should not be limited by the specific disclosure provided hereinbelow.

To achieve the above objects, an aspect of the present invention provides a composition for sustained inhibition of gene expression, containing an mRNA fragment of T7 RNA polymerase, plasmid DNA for self-amplification of the T7 RNA polymerase, and a DNA fragment encoding a gene expression inhibitor.

As used herein, the term “T7 RNA polymerase” refers to an RNA polymerase derived from the T7 bacteriophage that recognizes a promoter sequence unique to phage consisting of about 20 base pairs that catalyzes the synthesis of DNA from RNA in the 5′ to 3′ direction. T7 RNA polymerase has very high specificity for promoters and can only transcribe DNA downstream of the T7 promoter. Therefore, in the present invention, T7 RNA polymerase can bind and transcribe to the plasmid DNA having only the T7 promoter without reacting with other promoters in the cell, and can self-amplify the T7 RNA polymerase itself with high efficiency. Specifically, unlike eukaryotic RNA polymerase, which requires various intracellular cofactors, T7 RNA polymerase has the advantage of being able to induce transcription without an additional intracellular cofactor (M. Chamberlin, J. McGrath, L. Waskell, New RNA polymerase from Escherichia coli infected with bacteriophage T7, Nature 228 (1970) 227 to 231). Additionally, due to the absence of other RNA polymerase having specificity for the T7 promoter in the eukaryotic cell, the transcription system using the T7 promoter/T7 RNA polymerase is not interrupted by other transcription systems within the cell, and it does not interfere with other transcription systems. Meanwhile, since other promoters belonging to the same type of RNA polymerase III as T7 promoter (e.g., U1 promoter, H1 promoter, etc.) are already present in the cell, they do not have the above characteristic of not causing interference possessed by the T7 promoter, and the present invention is thus not suitable for controlling transcription on the transcription system. More specifically, the mRNA fragment of T7 RNA polymerase of the present invention may consist of a nucleotide sequence of SEQ ID NO: 1, but the mRNA fragment sequence is not limited thereto.

Additionally, the mRNA fragment of T7 RNA polymerase of the present invention may further include a 5′-cap structure. The 5′-cap structure refers to 7-methylguanosine present at the 5′-terminus of mRNAs of most eukaryotic cells and viruses, and it prevents the degradation of mRNA by ribonuclease (RNase) and may act as a ribosome binding site by binding to a translation initiation factor in the cytoplasm. Generally, cytoplasmic transcripts do not have the 5′-cap structure essentially possessed by nuclear transcripts, and thus they have a problem in that the translation efficiency by ribosome is decreased. As such, the present invention is characterized in that it uses the mRNA fragment of T7 RNA polymerase, and thereby, not only are intracellular stability and cytoplasmic translation efficiency increased, but also the limitations of the low nuclear membrane permeability of the plasmid DNA, which was previously used for the nucleus-dependent shRNA expression, are overcome, thereby allowing the initial T7 RNA polymerase to be effectively provided in the cytoplasm.

In an embodiment of the present invention, for the synthesis of T7 polymerase mRNA having a 5 ‘-cap structure, the dsDNA fragments having the sequence of the T7 promoter-T7 polymerase were first synthesized by a chemical synthesis method (Bioneer Corp, Korea), and for the T7 promoter-T7 polymerase DNA fragment, T7 polymerase mRNA having a 5′-cap structure was synthesized in vitro using the HiScribe T7 High Yield RNA Synthesis Kit (NEB).

In the present invention, the plasmid DNA is characterized in that T7 RNA polymerase is self-amplified in the cytoplasm by repetition of the expression loop of T7 RNA polymerase. Specifically, the plasmid DNA may consist of a T7 promoter, an internal ribosome entry site (IRES) domain, a gene encoding T7 RNA polymerase, a poly A tail, and a T7 termination sequence, which are operably linked. The IRES domain incorporated into the plasmid DNA may be a viral IRES, and can not only increase transcription stability of the plasmid DNA by producing an IRES-fused T7 polymerase transcript, but can also improve the low expression efficiency of T7 polymerase in the cytoplasm. Specifically, the T7 promoter may be comprised of a nucleotide sequence of SEQ ID NO: 2, the IRES domain may be comprised of a nucleotide sequence of SEQ ID NO: 3, the gene encoding T7 RNA polymerase may be comprised of a nucleotide sequence of SEQ ID NO: 4, and the T7 termination sequence may be comprised a nucleotide sequence of SEQ ID NO: 5, but these are not limited thereto.

In an embodiment of the present invention, T7 polymerase gene (GenBank Accession No. FJ881694.1) was inserted into the BamHI/XhoI restriction sites in the multi-cloning sites of the pT7CFE1-NHis plasmid (Thermo Fisher), and thereby a plasmid DNA (auto_T7pol plasmid) for self-amplification of T7 RNA polymerase was prepared, in which the T7 RNA polymerase gene was located downstream of the T7 promoter and the viral IRES domain while being simultaneously located upstream of the poly A tail and the T7 termination sequence.

As used herein, the “self-amplification” may be performed by the expression loop of T7 RNA polymerase due to the mRNA fragment of T7 RNA polymerase and plasmid DNA for self-amplification of the T7 RNA polymerase contained in the composition of the present invention. Specifically, the mRNA fragment of T7 RNA polymerase is translated by ribosomes in the cytoplasm to produce T7 RNA polymerase, and the produced T7 RNA polymerase recognizes the T7 promoter present in the plasmid DNA and performs transcription of T7 RNA polymerase. Then, the produced mRNA of T7 RNA polymerase is again translated by ribosomes in the cytoplasm to produce T7 RNA polymerase, and self-amplification of the T7 RNA polymerase is achieved by repetition of this entire process. Accordingly, the composition of the present invention is characterized in that it can maintain the T7 RNA polymerase self-amplified by the expression loop of T7 RNA polymerase at high concentration in the cytoplasm for a long period of time.

In an embodiment of the present invention, the cytoplasmic expression of T7 polymerase was examined over time by western blot in B16F10/RFP cells, which were treated with the 1,2-dioleoyl-sn-glycero-3-phosphocholine (DOPC) liposome transporter (auto_shRFP@LS) containing the plasmid DNA (auto_T7pol plasmid). As a result, it was confirmed that the concentration of the T7 polymerase was maintained in the cytoplasm at high concentration for at least 9 days (FIG. 3A).

The DNA fragment encoding the gene expression inhibitor of the present invention may be comprised of a T7 promoter, DNA encoding a gene expression inhibitor, and a T7 termination sequence, which are operably linked. The gene expression inhibitor may be any one selected from the group consisting of siRNAs, shRNAs, microRNAs, and aptamers, and specifically shRNA, but any one produced via transcription of the DNA fragment by the self-amplified T7 RNA polymerase can be included without limitation for the purpose of the present invention as long as it is used so as to inhibit the expression of a particular gene. In an embodiment of the present invention, siRFP (red fluorescent protein) was used as the gene expression inhibitor, but the gene expression inhibitor is not particularly limited thereto.

The term “small interfering RNA (siRNA)”, which is an RNAi-inducing material, refers to a short double-helix RNA strand consisting of about 20 to 30 nucleotides. Once siRNAs are injected into a cell, they target mRNAs with a complementary nucleotide sequence thereto and thereby inhibit the expression of the corresponding genes. The term “small hairpin RNA (shRNA)” refers to an artificial RNA molecule with a hairpin structure that can be used for inhibiting the expression of a target gene via the phenomenon of RNA interference. Since shRNA has a relatively low decomposition rate and turnover, it can be effectively used for RNAi. The term “microRNA” refers to a ribonucleic acid molecule having a length of about 22 nucleotides, and it is found in all eukaryotic cells. The microRNAs can inhibit the expression of a particular gene by binding to a target RNA transcript with a complementary sequence thereto thereby inhibiting translation of the transcript; by histone modification; or by inducing DNA methylation to a promoter of the target gene.

In an embodiment of the present invention, the long-term shRNA expression in the cytoplasm by the DOPC liposome transporter (auto_shRFP@ LS) and the presence of their conversion to siRNAs were evaluated. As a result, it was confirmed that siRFPs at high concentration were observed in the cells treated with the auto_shRFP@LS for at least 13 days, however, siRFPs at a significantly lower concentration were able to be measured in cells treated with auto(−)_shRFP@LS, which was used as the control, for only 10 days (FIGS. 3B and 3C).

The composition for sustained inhibition of gene expression of the present invention may be that which enables sustained long-term expression of the gene expression inhibitor by increasing the expression of cytoplasmic T7 RNA polymerase in a nucleus-independent manner Specifically, the sustained expression of the gene expression inhibitor may be sustained long-term production of shRNAs in the cytoplasm from the composition, but the expression is not limited thereto.

The existing method of shRNA expression in the nucleus using plasmid DNA with a nucleus-dependent promoter relies on cell membrane permeability, which has efficiency as low as 1%, and compared to that by cell membrane permeability, the efficiency of inhibiting gene expression is very low. Additionally, the shRNAs expressed in the nucleus can be converted by a dicer, etc. to mature siRNAs, which can participate in gene silencing, only when they are released into the cytoplasm. However, these exportin-5 transporters involved in the release of shRNAs into the cytoplasm are problematic because they become saturated by highly-concentrated shRNAs, thereby preventing even the release of microRNAs involved in other cellular functions.

As such, the present invention is characterized in that T7 RNA polymerase can be maintained in the cytoplasm at high concentration for a long period of time in a nucleus-independent manner through the expression loop of T7 RNA polymerase, which is performed by an mRNA fragment of T7 RNA polymerase with a 5′-cap structure and plasmid DNA for self-amplification of the T7 RNA polymerase, thereby enabling sustained long-term expression of the gene expression inhibitor.

As used herein, the term “target tissue” refers to a tissue in which gene expression is inhibited by the composition of the present invention, and specifically a cancer tissue, but the target tissue is not limited thereto.

Additionally, as used herein, the expression of “transferring a composition to a target tissue” may refer to the delivery of the composition of the present invention to the cells in the target tissue, and specifically, to the delivery of the composition of the present invention to the cells of the cancer tissue, but the delivery is not limited thereto.

To achieve the above objects, another aspect of the present invention provides a transporter of gene expression inhibitors, which includes the composition containing an mRNA fragment of T7 RNA polymerase; plasmid DNA for self-amplification of the T7 RNA polymerase; and a DNA fragment encoding a gene expression inhibitor. The details on T7 RNA polymerase, self-amplification, plasmid DNA, gene expression inhibitor, and composition are the same as described above.

As used herein, the term “transporter” may include without limitation any material which can enhance the delivery of the gene expression inhibitor into cells for the purpose of the present invention. Specifically, the transporter may be liposomes, polymer-based nanoparticles, dendrimers, and gold nanoparticles, and more specifically, cationic liposomes which are conventionally used for the delivery of genetic materials. The liposome may be DC-Chol/DOPE liposome, DMRIE/DOPE liposome, EDMPC/Chol liposome, GL-67/DOPE/DMPE/PEG liposome, or 1,2-dioleoyl-sn-glycero-3-phosphocholine (DOPC) liposome, but the liposome is not limited thereto.

Additionally, the transporter may include all of an mRNA fragment of T7 RNA polymerase; plasmid DNA for self-amplification of the T7 RNA polymerase; and a DNA fragment encoding a gene expression inhibitor, and the transporter may be delivered into a cell and perform the expression loop of T7 RNA polymerase, thereby increasing the expression of the T7 RNA polymerase in the cytoplasm and subsequent expression of the gene expression inhibitor (shRNA).

The transporter may be a liposome comprised of DOPC, and it may be prepared by mixing a composition, which comprises an mRNA fragment of T7 RNA polymerase, plasmid DNA for self-amplification of the T7 RNA polymerase, and a DNA fragment encoding gene-expression inhibitor; and DOPC in a weight ratio of 1:5 (w/w) to 1:10 (w/w), but the weight ratio is not limited thereto.

In an embodiment of the present invention, the transporter was prepared by mixing the oligonucleotide and DOPC in a weight ratio of 1:5, 1:7.5, and 1:10, respectively, under the conditions where the DNA fragment encoding a gene expression inhibitor: mRNA fragment of T7 RNA polymerase: plasmid DNA for self-amplification of the T7 RNA polymerase were in a 0.4:1:1 molar ratio. As a result, it was confirmed that when the weight ratio between oligonucleotide and DOPC was 1:10, most of the oligonucleotides were able to be loaded within the DOPC liposome transporter (FIG. 3A).

The transporter including the composition of the present invention may be able to effectively inhibit the expression of a target gene and protein produced from the target gene for a long period of time, compared to simple synthetic siRNA and the control group without plasmid DNA for self-amplification.

In an embodiment of the present invention, B16F10/RFP cells were treated with auto_shRFP@LS, and after a certain period of time, qRT-PCR was performed to examine the changes in the mRNA level of RFP. As a result, it was confirmed that the level of RFP mRNA was significantly reduced for at least 10 days, compared to those of the synthetic RFP siRNA and the control group, which was treated with auto(−)_shRFP@ LS (FIG. 4A).

Additionally, B16F10/RFP cells were treated with auto_shRFP@LS, and the level of RFP protein was examined after a certain period of time. As a result, it was confirmed that the level of RFP protein was significantly reduced for at least 10 days after the treatment with auto_shRFP@ LS, compared to the control group, which was treated with auto(−)_shRFP@ LS (FIG. 4B). Furthermore, even when the expression level of RFP protein was evaluated by flow cytometry, the treatment with auto_shRFP@ LS showed a significant decrease in the fluorescent signal for at least 10 days, compared to siRFP and the control group, which was treated with auto(−)_shRFP@LS (FIG. 4C).

Additionally, the transporter including the composition of the present invention may be able to effectively inhibit the expression of a target gene and the protein produced from the target gene for a long period of time, compared to the existing nucleus-dependent shRNA expression system.

In an embodiment of the present invention, the effect of auto_shRFP@ LS on the inhibition of gene expression compared to the plasmid DNA (pSuper_shRFP@LS) for the nucleus-dependent shRNA expression was evaluated. As a result, it was confirmed that the auto_shRFP@LS treatment showed a significant decrease in RFP mRNA level for at least 7 days (FIG. 5D. Additionally, even when the expression level of RFP protein was evaluated by flow cytometry, the cells treated with auto_shRFP@ LS showed a more effective decrease in protein expression even on the 4th day (FIG. 5E).

In the present invention, the transporter may be accumulated in a cancer tissue-specific manner. The cancer may be at least one selected from the group consisting of lung cancer, stomach cancer, colon cancer, breast cancer, bone cancer, pancreatic cancer, skin cancer, head cancer, head and neck cancer, melanoma, uterine cancer, ovarian cancer, colorectal cancer, small intestine cancer, rectal cancer, anal periphery cancer, fallopian tube carcinoma, endometrial cancer, uterine cervical cancer, vaginal cancer, vulvar cancer, Hodgkin's disease, esophageal cancer, lymphoma, bladder cancer, gallbladder cancer, endocrine cancer, prostate cancer, renal cancer, adrenal cancer, soft tissue sarcoma, urethral cancer, penile cancer, chronic or acute leukemia, lymphocytic lymphoma, renal cancer, ureteral cancer, kidney and renal pelvis cancer, blood cancer, brain cancer, central nervous system (CNS) tumor, spinal cord tumor, brain stem glioma, and pituitary adenoma, but the cancer is not limited thereto.

In an embodiment of the present invention, mice were inoculated with B16F10 cancer cells, administered with the fluorescently-labeled transporter (Cy5.5-auto_shRFP@LS) of the present invention, and major organs and tumor tissues were removed from the mice and the accumulation level of Cy5.5-auto_shRFP@ LS was evaluated. As a result, it was confirmed that the control group, where the fluorescently-labeled Cy5.5-siRFP was administered, showed the highest accumulation level in the kidneys, whereas when Cy5.5-auto_shRFP@ LS nanoparticles were administered, the highest accumulation level was observed in the tumor tissue (FIG. 6). That is, these results show that the transporter including the composition of the present invention can effectively perform selective, sustained long-term delivery of a gene expression inhibitor to cancer tissue by the EPR effect.

Additionally, mice were inoculated with B16F10/RFP cancer cells and administered with auto_shRFP@LS, and the fluorescence intensity of RFP was observed. As a result, it was confirmed that the fluorescence intensity and the relative expression level of RFP mRNA were significantly inhibited in the cancer tissue with time (about 6 days) (FIGS. 7A to 7E). That is, these results show that the transporter including the composition of the present invention can effectively inhibit a target gene in cancer tissue for even a period of 6 days or more.

To achieve the above objects, still another aspect of the present invention provides a method for preparing the transporter of a gene expression inhibitor. The T7 RNA polymerase, self-amplification, plasmid DNA, gene expression inhibitor, composition, and transporter are the same as described above.

Specifically, the above preparation method may include a method for preparing a transporter of a gene expression inhibitor, which includes: preparing each of an mRNA fragment of T7 RNA polymerase, plasmid DNA for self-amplification of the T7 RNA polymerase, and a DNA fragment encoding a gene expression inhibitor; and mixing the composition, which comprises the mRNA fragment of T7 RNA polymerase, plasmid DNA for self-amplification of the T7 RNA polymerase, and DNA fragment encoding gene-expression inhibitor; with DOPC. The method may further include removing free oligonucleotides by filtration after the step of mixing, but the method is not limited thereto.

In the above step of mixing, the oligonucleotides and DOPC may be mixed in a ratio of 1:5 (w/w) to 1:10 (w/w) for preparation, and more specifically in a 1:10 (w/w) ratio, but the mixing ratio is not limited thereto. Additionally, the DNA fragment encoding a gene expression inhibitor, mRNA fragment of T7 RNA polymerase, and plasmid DNA for self-amplification of the T7 RNA polymerase may be mixed in a 0.4:1:1 molar ratio, but the molar ratio is not particularly limited thereto.

### Advantageous Effects of the Invention

The composition of the present invention and the liposome transporter of the present invention including the composition can improve the expression of shRNA in the cytoplasm through self-amplification of nucleus-independent, sustained self-amplification of T7 RNA polymerase, and deliver them in a cancer tissue-specific manner Therefore, the composition and the liposome transporter of the present invention can be utilized for use in treating chronic diseases that require reduced frequency of administration and long-term inhibition of gene expression.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A shows a schematic diagram illustrating the mechanisms of pT7/T7 polymerase self-amplification of T7 polymerase and expression of shRNA in the cytoplasm by expressed T7 polymerase according to the present invention, and FIG. 1B shows a schematic diagram illustrating the pT7/shRNA DNA fragment that enables cytoplasmic expression of shRNA by T7 polymerase, and a predicted result of a two-dimensional structure of shRNA being expressed.

FIG. 2 shows a vector map of pT7CFE1-NHis plasmid (Thermo Fisher) which was used for the preparation of plasmid DNA (auto_T7pol plasmid) for the self-amplification of T7 RNA polymerase.

FIG. 3A shows the results of 1.5% agarose gel electrophoresis, in which, for the 3 kinds of oligonucleotides that constitute the sustained expression system of shRNA in the cytoplasm, pT7/shRNA DNA template, 5′-capped T7pol mRNA, and auto_T7pol plasmid DNA were fixed in a molar ratio of 0.4:1:1; and a certain amount of each of the 3 kinds of oligonucleotides and DOPC liposome were bound in a certain binding ratio (1:0, 1:5, 1:7.5, and 1:10 (w/w)). In the binding ratio of 1:10 (w/w), it can be seen that most of the oligonucleotides can form a complex with DOPC liposome.

FIG. 3B shows the measurement results of diameters of oligonucleotides/DOPC complexes (auto_shRFP@LS) including the RFP shRNA (shRFP) sequence capable of silencing the RFP, obtained using dynamic light scattering, and the diameter at the optimal binding ratio (1:10 (w/w)) is shown to be 31.4 nm.

FIG. 3C shows the results of cytotoxicity of auto_shRFP@LS in B16F10/RFP cells examined using MTT assay. When the cytotoxicity of auto_shRFP@ LS was examined after 24 hours or 48 hours, no cytotoxicity was observed even at a high concentration of about 60 μg/mL.

FIG. 3D shows the results of cell permeability of Cy5.5-labeled auto_shRFP@LS in B16F10 cells using flow cytometry. These results were obtained one hour after transfection and it was confirmed that Cy5.5-labeled auto_shRFP@ LS was effectively permeated into most cells.

FIG. 3E shows that Cy5.5-labeled auto_shRFP@LS was effectively permeated into the cytoplasm using a confocal fluorescent microscope.

FIG. 3F shows TNF-α release and INF-α release, by an innate immune response, in human peripheral blood mononuclear cells (PBMC) after 4 hours or 24 hours of treatment with various kinds of immunostimulatory materials (i.e., PBS (5 μM), auto_shRFP@ LS (15 μg/mL), lipopolysaccharides (55 ng/mL), or CpG oligodeoxynucleotides (6 μM)). In particular, lipopolysaccharides were used as a positive control for TNF-α, and CpG oligodeoxynucleotides were used as a positive control for INF-α.

FIG. 4A shows the results of the expression level of T7 polymerase capable of self-amplification in the cytoplasm of B16F10/RFP cells treated with auto_shRFP@LS, measured according to time using a T7 polymerase antibody. As the control group, the expression level of T7 polymerase was measured in cells treated with auto(−)_shRFP@LS where the auto_T7pol plasmid of FIG. 1A is omitted. The auto_shRFP@LS capable of self-amplification showed an expression of high-concentration T7 polymerase for at least 9 days, whereas auto(−)_shRFP@ LS incapable of self-amplification showed a lower expression efficiency of T7 polymerase.

FIG. 4B shows a standard curve between the concentration of synthetic RFP siRNA (siRFP) guide strands and the Cycle Threshold (CT) values using the siRFP-specific small RNA TaqMan assay. The use of such a standard curve enables the measurement of intracellular siRNA concentration by the measurement of CT values.

FIG. 4C shows the results of quantification of shRFPs, which were produced in B16F10/RFP cells treated with auto_shRFP@ LS and converted to siRNAs in the form of active molecules by RNAi machinery, using the siRFP-specific small RNA TaqMan assay. According to the measurement results of the amount of intracellular siRFPs based on the standard curve provided in FIG. 4B and the CT values exhibited by intracellular siRFPs, it is shown that auto_shRFP@ LS enables the maintenance of high-concentration siRFPs in cells for at least 13 days, whereas auto(−)_shRFP@LS incapable of self-amplification of T7 polymerase produces siRFPs at a significantly lower efficiency.

FIG. 5A shows the results of the mRNA level of RFP gene (i.e., target gene), which decreased with time in B16F10/RFP cells treated with auto_shRFP@LS, quantified by qRT-PCR. As the control groups, siRFP@LS (prepared in the form of DOPC liposome using the method of Example 2), which used synthetic RFP siRNA, and auto(−)_shRFP@LS were used. According to the observation, auto_shRFP@LS induced an apparent decrease in the level of RFP mRNA for at least 10 days, whereas siRFP@LS induced a decrease in the level of RFP mRNA for only 4 days. Meanwhile, auto(−)_shRFP@LS induced an apparent decrease in the level of RFP mRNA for at least 10 days, but the decrease efficiency was observed to be significantly lower than that induced by auto_shRFP@LS.

FIG. 5B shows the results of the level of RFP protein, which decreased with time in B16F10/RFP cells treated with auto_shRFP@ LS, measured by western blot using an RFP antibody. According to the observation, auto_shRFP@LS induced an apparent decrease in the level of RFP protein for at least 10 days, whereas siRFP@LS, which was used as the control group, induced an apparent decrease in the level of RFP protein for only 3 days and the RFP protein level returned to the original level from the 4th day.

FIG. 5C shows the measurement results of the fluorescence intensity of RFP (i.e., a fluorescent protein), which was inhibited with time in B16F10/RFP cells treated with auto_shRFP@LS, quantified by flow cytometry. According to the observation, auto_shRFP@LS showed an apparent decrease in the fluorescence intensity of RFP for at least 10 days, whereas siRFP@LS, which was used as a control group, showed no such decrease in the fluorescence intensity of RFP from the 7th day. Additionally, auto(−)_shRFP@LS, which was also used as a control group, showed a decrease in the fluorescence intensity of RFP for 7 days, but the decrease efficiency was observed to be significantly lower than that induced by auto_shRFP@ LS.

FIG. 5D shows the comparison results of the ability of reducing the mRNA level of RFP in B16F10/RFP cells between pSuper_shRFP plasmid@LS, which is capable of nucleus-dependent shRNA expression based on the HI promoter, and auto_shRFP@LS, which is capable of nucleus-independent cytoplasmic shRNA expression. According to the observation, pSuper_shRFP plasmid@LS induced a decrease of the mRNA level of RFP for about 3 days, whereas auto_shRFP@LS induced an apparent decrease of the mRNA level of RFP for at least 7 days.

FIG. 5E shows the results of decrease of fluorescence intensity of RFP after treating B16F10/RFP cells respectively with pSuper_shRFP plasmid@LS and auto_shRFP@LS, measured by flow cytometry. According to the observation, pSuper_shRFP plasmid@LS induced the decrease of fluorescence intensity of RFP for only 3 days, whereas auto_shRFP@ LS induced the decrease of fluorescence intensity of RFP for a longer period of time.

FIG. 6 shows the measurement results of fluorescence intensity of Cy5.5 in organs and cancer tissues, which were removed from mice 24 hours after intravenous injection of fluorescently-labeled Cy5.5-labeled auto_shRFP@LS. According to the observation, Cy5.5-labeled siRFP@LS, which was used as the control group, was mostly accumulated in the kidneys, whereas Cy5.5-labeled auto_shRFP@LS was accumulated in both the kidneys and livers, and predominantly in cancer tissues.

FIG. 7A shows the results of decrease of fluorescence intensity of RFP protein according to time (0, 3, 4, 5, and 6 days) in cancer tissues of B16F10/RFP tumor-bearing mice after intravenous injection of auto_shRFP@LS. The intravenous injection was performed only once, and for the control group, PBS buffer and siRFP@LS were intravenously injected.

FIG. 7B shows the quantitative results of FIG. 7A. In mice of the control group where PBS buffer was intravenously injected, cancer tissue grew with time, and proportionally, the fluorescence intensity of RFP in cancer tissue increased (FIG. 6B). In mice where siRFP@LS was intravenously injected, the fluorescence intensity of RFP was shown to be inhibited in cancer tissue for about 3 days, but the fluorescence intensity of RFP was shown to increase again from the 4th day. However, in mice where auto_shRFP@LS was intravenously injected, it was observed that the fluorescence intensity was inhibited throughout the 6 day observation period.

FIG. 7C shows the measurement results of fluorescence intensity of RFP in cancer tissue removed from the mice of FIG. 7A, 6 days after the intravenous injection. The fluorescence intensity of RFP in mice, where siRFP@LS was intravenously injected, was similar to that in cancer tissue removed from the mice where PBS buffer was intravenously injected. However, the fluorescence intensity of RFP in cancer tissue, which was removed from the mice where auto_shRFP@ LS was intravenously injected, was shown to be significantly reduced.

FIG. 7D shows the results of immunohistochemistry analysis performed using an RFP antibody with regard to cancer tissue removed from the mice of FIG. 7C. According to the observation, the cancer tissues removed from the mice where each of PBS buffer and siRFP@ LS was intravenously injected showed the presence of a large amount of RFP protein (stained in brown), whereas the cancer tissue removed from the mice where auto_shRFP@LS was intravenously injected showed an apparent decrease of RFP protein.

FIG. 7E shows the results of the relative amount of mRNA of RFP in cancer tissue removed from the mice of FIG. 7C, quantified by qRT-PCR.

FIG. 8 shows the results of the relative difference in molecular weight between 5′-capped T7pol mRNA and 5′-cap-free T7pol mRNA confirmed by agarose gel electrophoresis. The relative difference in mobility of these bands occurs due to the difference in molecular weight of the 5′-cap moiety.

FIG. 9 shows a schematic diagram of pSuper_shRFP plasmid (i.e., an H1 promoter-based nucleus-dependent shRNA expression system) and the predicted results of a two-dimensional structure of the shRFP sequence being expressed.

FIG. 10 shows the results of TNF-α and INF-α levels measured using blood samples of C57BL/6J mice, where auto_shRFP@LS was intravenously injected, collected 1 hour or 24 hours after the intravenous injection so as to examine proinflammatory cytokine induction. As the control group, siRFP@Lipofectamine and PBS buffer were used.

### DETAILED DESCRIPTION OF THE INVENTION

Hereinafter, the present invention will be described in more detail with reference to the following Examples. However, these Examples are for illustrative purposes only and the scope of the invention is not limited by these Examples.

### Example 1. Design and Preparation of pT7/T7 Polymerase System Capable of Sustained Expression of shRNA in Cytoplasm

1-1. Design of Auto_T7pol Plasmid Capable of Sustained Cytoplasmic Self-Amplification of T7 Polymerase

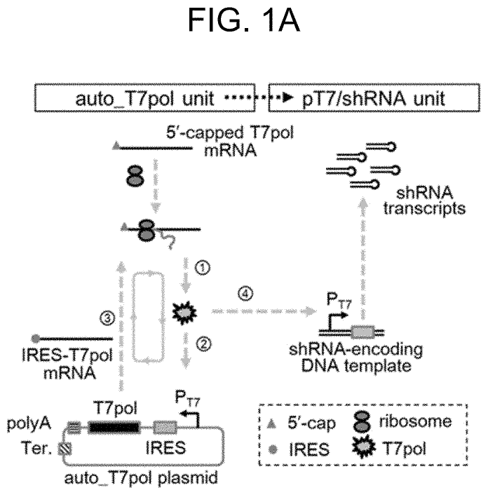

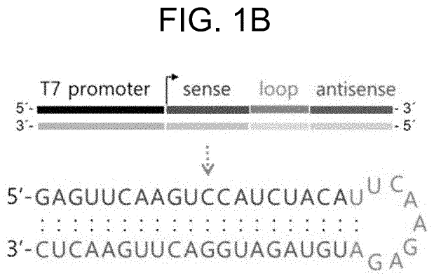

To insert T7 polymerase gene (GenBank Accession No. FJ881694.1) using BamHI/XhoI sites of the multicloning site of pT7CFE1-NHis plasmid (Thermo Fisher), first, T7 polymerase gene was amplified by PCR method using chromosomal DNA of E. coli BL21 (DE3) (Novagen, USA) as a template. In particular, both termini include BamHI/XhoI site sequences. The amplified PCR product was treated with BamHI/XhoI restriction enzymes and inserted into the pT7CFE1-NHis plasmid using T4 DNA ligase. In particular, T7 polymerase gene is located downstream of T7 promoter and viral IRES element provided by the pT7CFE1-NHis plasmid, while simultaneously being located upstream of polyA sequence and T7 termination sequence thereof (T7 promoter/viral IRES element/T7 polymerase/polyA tail/T7 termination). The design of auto_T7pol plasmid prepared in this Example is shown in FIG. 1A.

1-2. Preparation of DNA Template for Cytoplasmic shRNA Expression and 5′-Capped T7pol mRNA for Supplying First T7 Polymerase

To prepare a pT7/shRNA DNA template to which the T7 polymerase gene, which was expressed in Example 1-1, is able to bind and express shRNA, SEQ ID NOS: 6 and 7 (Table 1) were synthesized by a chemical synthesis method (outsourced to Bioneer Corporation, Korea). T7 promoter was indicated in lower case, RFP siRNA sequence in bold, and the loop sequence of hairpin in underline, respectively.

TABLE 1

| | |
|---|---|
| SEQ ID NO: 6 | 5'-taatacgactcactataggGAGTTCAAGTCCATCT |
| | ACATTCAAGAGATGTAGATGGACTTGAACTC-3' |
| SEQ ID NO: 7 | 5'-GAGTTCAAGTCCATCTACATCTCTTGAATGTAGAT |
| | GGACTTGAACTCtatagtgagtcgtatta-3' |

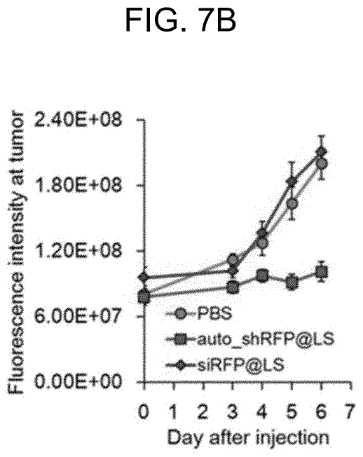

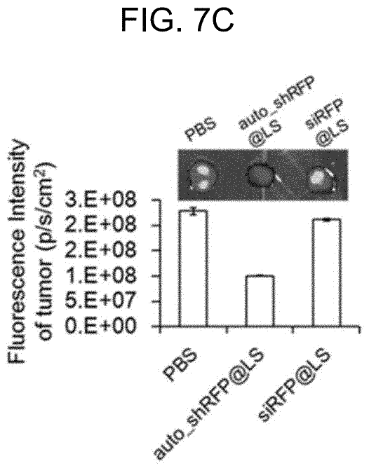

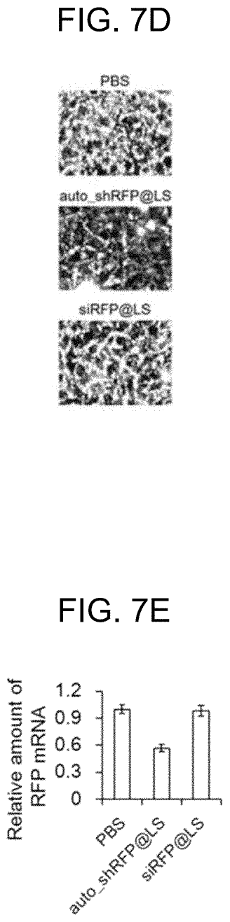

For the initiation of self-amplification of T7 polymerase in the auto_T7pol plasmid of Example 1-1, it is necessary to supply the first T7 polymerase, and in the present invention, 5′-capped T7pol mRNA was used so as to solve the problem of low nuclear membrane permeability. For the synthesis of 5′-capped T7pol mRNA, a dsDNA template having a T7 promoter-T7 polymerase sequence was first synthesized by a chemical synthesis method (outsourced to Bioneer Corporation, Korea). With regard to the T7 promoter-T7 polymerase DNA template, it was possible to synthesize 5′-capped T7pol mRNA in vitro using the HiScribe T7 High Yield RNA Synthesis Kit (NEB). More specifically, during the in vitro transcription reaction, anti-reverse cap analog (ARCA) (also called 3′-O-Me-7mG(5′)ppp(5′)G cap analog) was added along with four kinds of standard NTPs, in which the molar ratio of ARCA:GTP was 4:1. The dsDNA template was removed by adding DNA nuclease thereto, and the remaining 5 ‘-capped T7pol mRNA was purified using a LiCl extraction method and an ethanol precipitation method. The 5’-cap-free T7pol mRNA (i.e., the control group) was synthesized based on the same T7 promoter-T7 polymerase DNA template using the MEGAscript T7 Transcription Kit (Thermo Fisher Scientific). The difference in molecular weight between the synthesized 5′-capped T7pol mRNA and 5′-cap-free T7pol mRNA was confirmed by performing agarose gel electrophoresis (FIG. 7).

1-3. Preparation of pSuper_shRFP Plasmid for Nucleus-Dependent shRNA Expression Based on H1 Promoter (Used as Control Group)

To prepare pSuper_shRFP plasmid capable of expressing shRNA in the nucleus by H1 promoter, SEQ ID NOS: 8 and 9 (Table 2) including the BglII and XhoI restriction enzyme sequences at both termini were synthesized by a chemical synthesis method (outsourced to Bioneer Corporation, Korea). RFP siRNA sequence was indicated in underline and the sequence of loop portion of hairpin was indicated in lower case.

TABLE 2

| | |
|---|---|
| SEQ ID NO: 8 | 5'-GGATCCCCGAGTTCAAGTCCATCTACAttcaagag |
| | aTGTAGATGGACTTGAACTCTTTTTCTCGAG-3' |
| SEQ ID NO: 9 | 5'-CTCGAGAAAAAGAGTTCAAGTCCATCTACAtctct |
| | tgaaTGTAGATGGACTTGAAGTCGGGGATCC-3' |

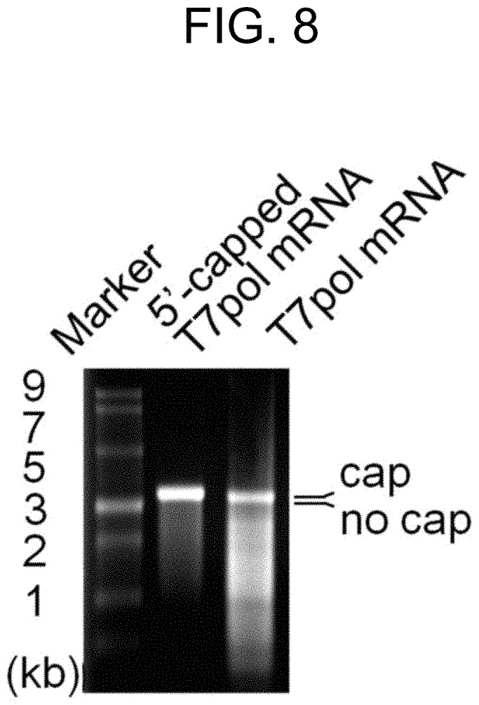

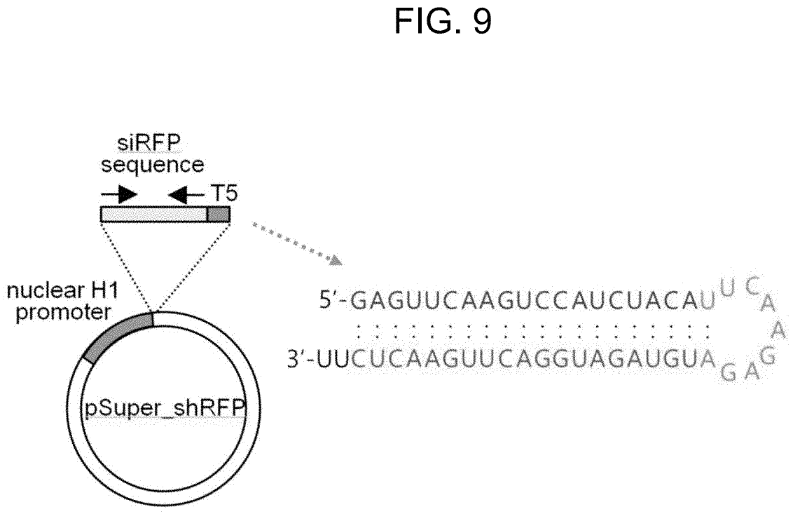

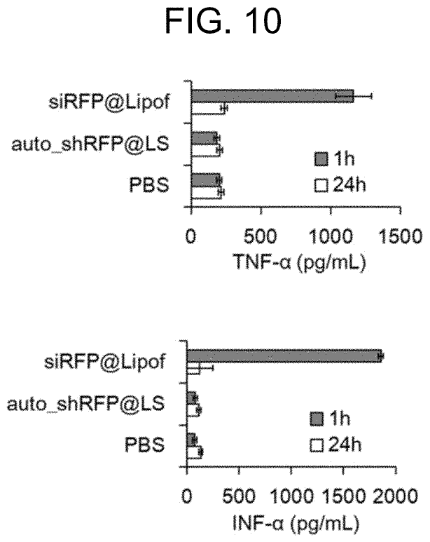

The dsDNA was treated with BglII and XhoI restriction enzymes and inserted into the BglII/XhoI sites of the multicloning site of pSuper_basic plasmid (OligoEngine) using T4 DNA ligase (FIG. 8). The two-dimensional structure of the shRFP hairpin, which is expressed in the nucleus by the pSuper_shRFP plasmid, is shown in FIG. 8.

### Example 2. Preparation of DOPC Liposome Transporter Loaded with Sustained Cytoplasmic Expression System for shRNA

To prepare DOPC liposomes loaded with a sustained cytoplasmic expression system for shRNA, first, DOPC lipid (26.5 μg) was mixed with the auto_T7pol plasmid (1.875 μg) prepared in Example 1-1; pT7/shRNA DNA template (0.15 μg) and 5′-capped T7pol mRNA (0.625 μg) prepared in Example 1-2; and an excess amount of t-butanol. Tween 20 was added to the mixture under the condition where Tween 20: oligonucleotides/DOPC is 1:19 (w/w), and the resultant was lyophilized in an acetone/dry ice bath so as to remove the organic solvent. Then, a 0.9% saline solution was added to the lyophilized mixture, and free oligonucleotides which were not loaded in the liposomes were removed using the amino μLtracentrifuge filter (30K MWCO, Millipore). The amount of free oligonucleotides that passed through a filter was measured using the Nanodrop spectrophotometer, and the concentration of the oligonucleotides loaded in the liposomes (the oligonucleotide/DOPC liposome complex) was shown to be 150 μg/mL.

### Example 3. Examination of Biophysicochemical Properties of DOPC Liposome Transporter Loaded with Sustained Cytoplasmic Expression System for shRNA

It was confirmed by agarose gel electrophoresis that most oligonucleotides can be loaded in the DOPC liposomes when the molar ratio of total oligonucleotides:DOPC is within the range of 1:10 (w/w) under the condition where the molar ratio of pT7/shRNA DNA template: 5′-capped T7pol mRNA:auto_T7pol plasmid DNA is 0.4:1:1 (FIG. 2A). The particle size of the oligonucleotides/DOPC liposome complex (auto_shRNA@LS) was measured using the dynamic light scatter, and it was shown to have a diameter of 31.4 nm (FIG. 2B).

As can be seen in FIG. 2C, the intracellular biocompatibility of auto_shRFP@LS was examined by the MTT assay (Choi, Y. H.; Liu, F.; Kim, J. S.; Choi, Y. K.; Park, J. S.; Kim, S. W. J. Control. Rel. 1998, 54, 39 to 48). Specifically, the B16F10/RFP cells in an exponential growth phase were grown in a 96-well plate to a density of 20,000 cells/well. Each well was treated with the auto_shRFP@LS prepared in Example 2 at various concentrations, and the cells were cultured for 24 or 48 hours. MTT solution (0.5 mg/mL) was added to each well in an amount of 200 μL and reacted for 4 hours. Then, DMSO (200 μL) was added thereto and reacted for 10 minutes, and the absorbance at 570 nm was measured by ELISA. As a result, the auto_shRFP@LS nanoparticles showed excellent biocompatibility in B16F10/RFP cells when cultured for both 24 and 48 hours up to the concentration of 60 μg/mL (FIG. 2C).

To examine the intracellular permeability of the auto_shRFP@ LS nanoparticles, auto_shRFP@ LS nanoparticles to which Cy5.5 phosphors were bound were treated on B16/F10 cells, and the resultant cells were analyzed after an hour by flow cytometry and confocal fluorescent microscope (FIGS. 2D and 2E). As a result of the flow cytometry, it was observed that a majority of the auto_shRFP@LS nanoparticles to which Cy5.5 phosphors were bound permeated into the cells after an hour. From the confocal fluorescent microscope analysis, it was observed that a majority of the auto_shRFP@LS nanoparticles to which Cy5.5 phosphors were bound permeated into the cytoplasm after an hour.

To examine the innate immunogenicity that can be caused by the oligonucleotides or the oligonucleotide/DOPC transporter of the present invention, auto_shRFP@ LS nanoparticles were treated on human PBMC cells and the TNF-α and INF-α induction were examined after 4 hours or 24 hours (FIG. 2F). As a result, the auto_shRFP@LS nanoparticles did not cause TNF-α induction, unlike the lipopolysaccharides which caused TNF-α induction. The INF-α induction was also examined, and it was found that auto_shRFP@LS nanoparticles hardly caused INF-α induction, while CpG oligodeoxynucleotides released a large amount of INF-α. These results demonstrate that the auto_shRFP@ LS nanoparticles do not stimulate innate immunogenicity and thus it is possible to intravenously inject the auto_shRFP@ LS nanoparticles during clinical application.

### Example 4. Experiment on Sustained Expression of T7 Polymerase Using Modified pT7/T7 Polymerase System

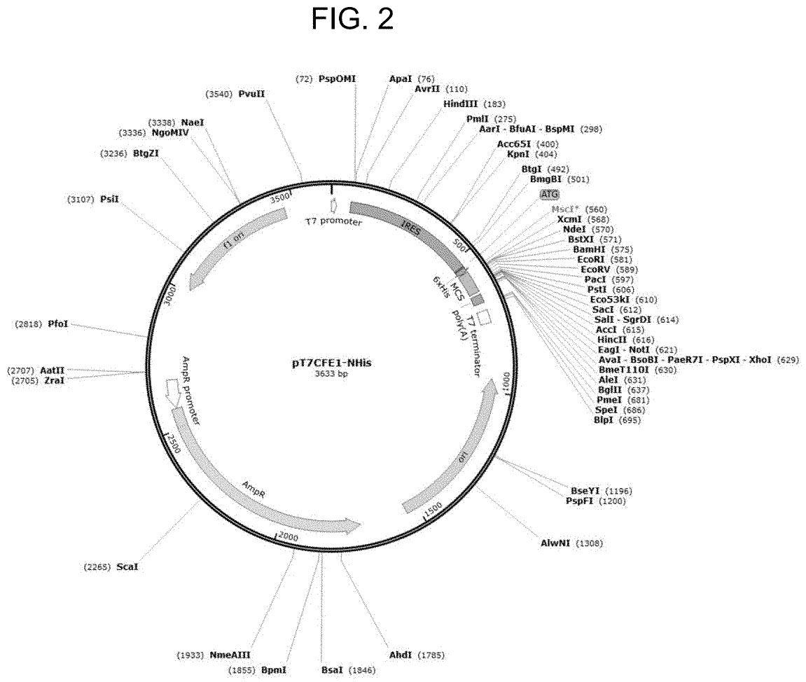

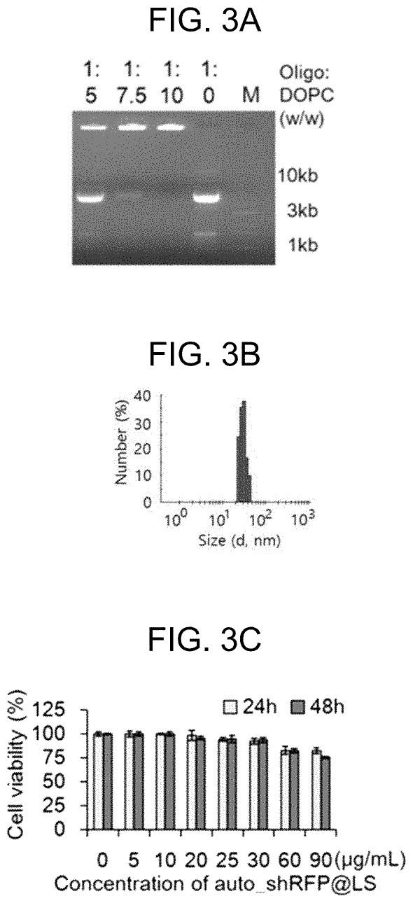

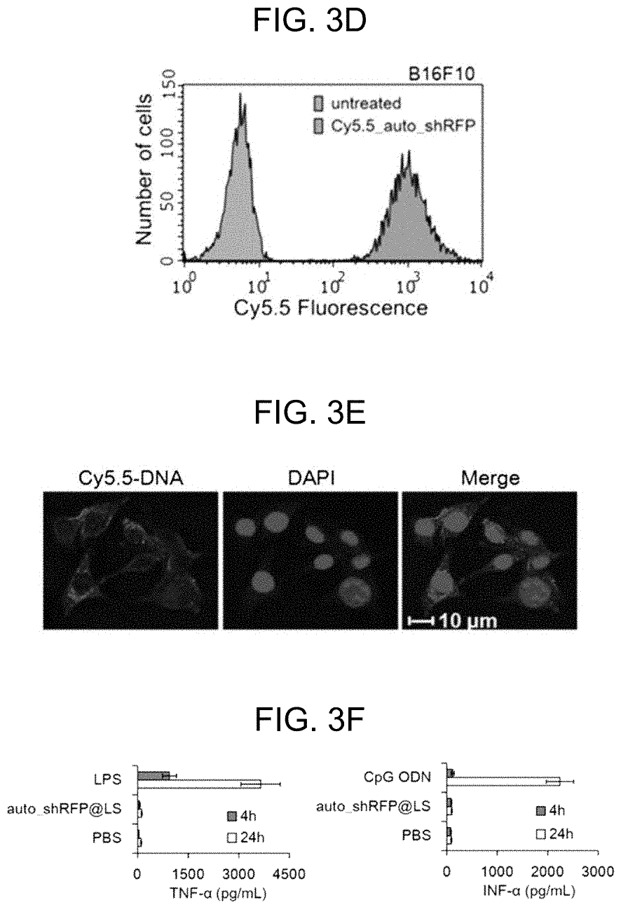

The cytoplasmic expression of T7 polymerase in B16F10/RFP cells treated with auto_shRFP@ LS according to time (1, 3, 5, 7, and 9 days) was examined by western blot analysis using a T7 polymerase antibody (FIG. 3A). The cells treated with auto_shRFP@ LS for the specified time as described above were lysed with a RIPA buffer containing a cocktail protease inhibitor and centrifuged to obtain protein samples. These samples were subjected to 10% SDS-PAGE electrophoresis and transferred into PVDF membranes. To prevent non-specific staining, these membranes were blocked in the presence of 0.2% Tween-20 and 5% dry milk, subjected to primary staining with a T7 polymerase antibody, secondary staining with a secondary antibody to which HRP was bound, and developed using chemiluminescence.

As can be seen in FIG. 3A, T7 polymerase was maintained in the cytoplasm at high concentration for at least 9 days, and this was made possible because the 5′-capped T7 polymerase mRNA and the IRES element were bound to the pT7/T7 polymerase system, thus enabling efficient self-amplification. The 5′-capped T7 polymerase mRNA, which was transfected to cells at the early stage, can express the T7 polymerase by translational machinery containing cytoplasmic ribosomes, and the T7 polymerase binds to the T7 promoter of the auto_T7pol plasmid, thereby repeatedly producing cytoplasmic T7 polymerase transcripts.

As a result, in the cells used as the control group, where auto(−)_shRFP@LS that lacks auto_T7pol plasmid was treated, the cytoplasmic self-amplification of T7 polymerase was impossible, and thus the concentration of T7 polymerase expressed in the cytoplasm was relatively low (FIG. 3A). Additionally, when the IRES element was removed from the auto_T7pol plasmid, the amount of cytoplasmic T7 polymerase was observed to be similar to that of auto(−)_shRNA @ LS. These results demonstrate that the modified pT7/T7 polymerase system including the IRES element and the 5′-capped T7 polymerase mRNA can perform long-term (for at least 9 days) sustained expression of T7 polymerase in the cytoplasm.

### Example 5. Examination of Long-Term Cytoplasmic Expression of shRNAs by Auto_shRFP@LS and their Conversion to siRNAs

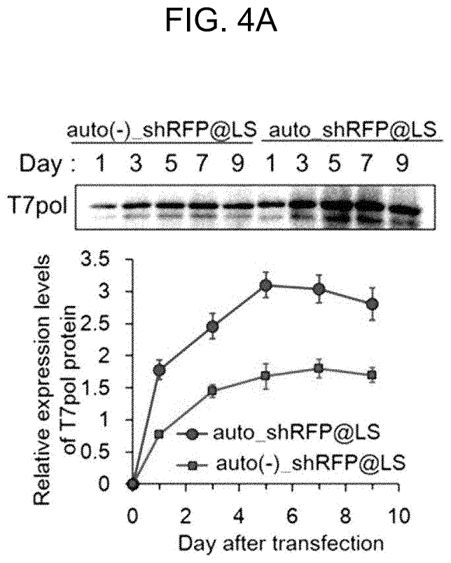

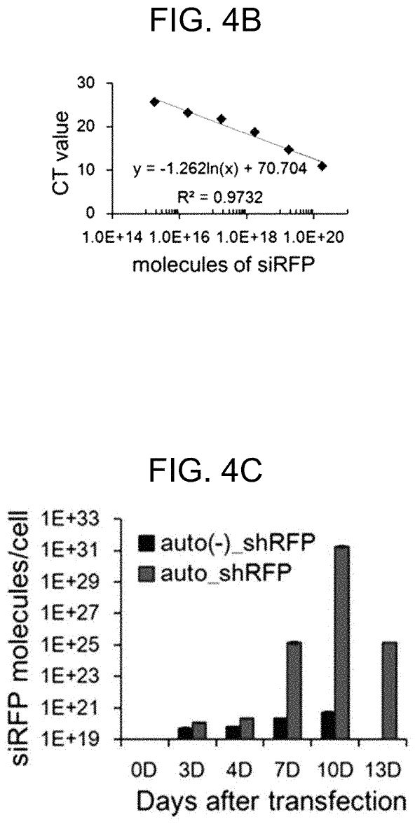

It was examined whether the T7 polymerase, whose concentration is maintained for a long period of time in the cytoplasm at high concentration as a result of the self-amplification of T7 polymerase by auto_T7pol plasmid and 5′-capped T7 polymerase mRNA, repeatedly binds to the T7 promoter of the pT7/shRNA DNA template, and thus the sustained long-term shRNA expression is maintained, and these shRNAs are converted into the form of siRNAs that are actually involved in gene silencing, by the small RNA-specific TaqMan assay (FIGS. 4B and 4C).

First, to measure the amount of siRNAs present in the cytoplasm, standard curves were plotted with regard to the guide siRNA of the synthetic RFP siRNA using the Custom TaqMan® Small RNA assay kit (FIG. 4B). Specifically, with regard to the guide siRNA of the synthetic RFP siRNA, the cDNA of the guide siRNA of the synthetic RFP siRNA was synthesized using the custom-made siRFP-specific stem-loop TR primers, and quantitative PCR (qPCR) was performed using custom-made siRFP-specific primers, to which a fluorescent dye was attached, with regard to the produced cDNA, and thereby the exponential correlation between the guide siRNA of the synthetic RFP siRNA and its amplification patterns was drawn. The results are illustrated in FIG. 4B. Based on these standard curves, the amount of RFP siRNA quantified relative to the RNA extracted from the B16F10/RFP treated with auto_shRFP@LS is shown in FIG. 4C. More specifically, the cDNA of the guide siRNA was synthesized from the extracted RNA (about 2 μg) using the custom-made siRFP-specific stem-loop TR primers, and quantitative PCR (qPCR) was performed using custom-made siRFP-specific primers, to which a fluorescent dye was attached, with regard to the produced cDNA, and thereby the exponential correlation between the guide siRNA of the synthetic RFP siRNA and its amplification patterns was drawn. The drawn exponential correlation was applied to the standard curves of FIG. 4B, and the measurement results of the amount of cytoplasmic RFP siRNA are shown in FIG. 4C.

As a result, it was possible to observe siRFPs at high concentration in the cells treated with auto_shRFP@LS for at least 13 days. However, in the cells treated with auto(−)_shRFP@LS (control group), it was possible to measure only the siRFPs at a much lower concentration for only 10 days. These results demonstrate that the cytoplasmic shRNA expression using the auto_shRFP@LS is being performed over a long period of time, and the thus-produced shRNA molecules are effectively converted to siRNA molecules by the cytoplasmic RNAi machinery containing dicers.

### Example 6. Examination of In Vitro Gene Silencing by Auto shRNA @LS Nanoparticles

B16F10/RFP cells were treated with auto_shRFP@LS (final concentration: 15 μg/mL) and qRT-PCR was performed after 3, 4, 7, 10, and 13 days, thereby measuring the changes in the amount of mRNA of RFP (i.e., target gene). Specifically, qRT-PCR was performed so as to measure the amount of intracellular mRNA of RFP gene using primers that can match with the RFP mRNA (a forward primer (5′-GCGTGATGAACTTCGAGGA-3′: SEQ ID NO: 10) and a reverse primer (5′-CAATAGTGATGACCTGGCCGT-3′: SEQ ID NO: 11)); primers that can match with (3-actin for the experiment of the control group (a forward primer (5′-AGAGGGAAATCGTGCGTGAC-3′: SEQ ID NO: 12) and a reverse primer (5′-CAATAGTGATGACCTGGCCGT-3′: SEQ ID NO: 13)) under the following conditions: 20 cycles of denaturation at 95° C. for 30 seconds; annealing at 51° C. for 30 seconds; and elongation at 72° C. for 30 seconds. As the control group, the cells were treated with siRFP@LS (final concentration: 50 nM siRFP) loaded with synthetic RFP siRNA, and after the lapse of the same period of time, the changes in the amount of RFP mRNA were measured.

As a result, when the cells were treated with the siRFP@LS loaded with synthetic RFP siRNA, the decrease in the amount of RFP mRNA was observed for 4 days, and on the 6^{th} day, the amount of RFP mRNA was recovered to a normal level. However, when the cells were treated with auto_shRFP@LS, the decrease in the amount of RFP mRNA was observed for at least 10 days. When the cells were treated with auto(−)_shRFP@LS (control group), the decrease in the amount of RFP mRNA was observed for 10 days, but the degree of decrease was significantly lower compared to when the cells were treated with auto_shRFP@LS (FIG. 4A). That is, these results demonstrate that auto_shRFP@LS continuously produces shRFP and thus can effectively decompose RFP mRNA for at least 10 days.

Additionally, gene silencing of auto_shRNA@LS nanoparticles was examined by western blot analysis using an anti-RFP antibody. B16F10/RFP cancer cells were treated with auto_shRFP@LS (final concentration: 15 μg/mL) and the changes in the amount of RFP protein were measured after 0, 3, 4, 7, and 10 days. As the control group, the cells were treated with siRFP@LS (final concentration: 50 nM siRFP), and after the lapse of the same period of time, the changes in the amount of RFP mRNA were measured.

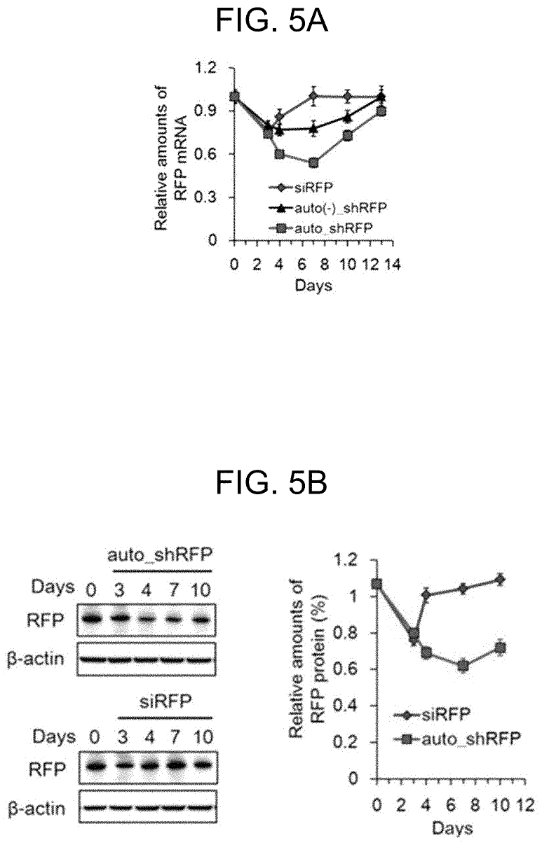

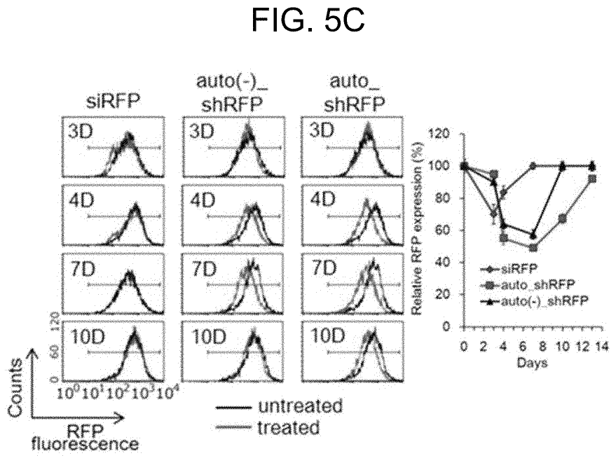

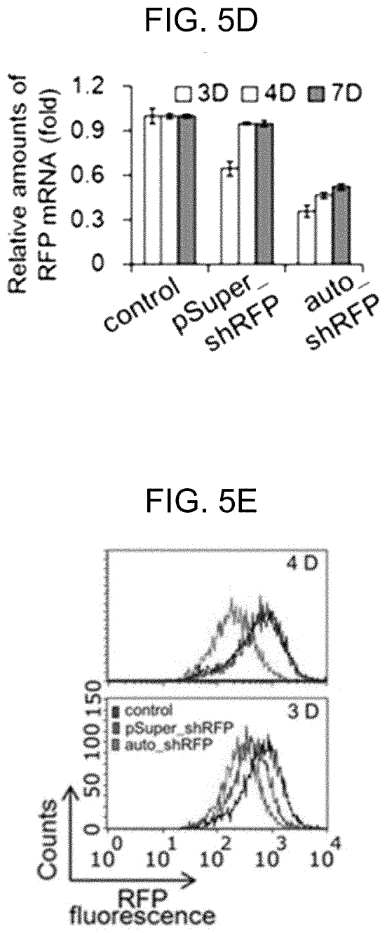

As a result, when the cells were treated with siRFP@LS, the decrease in the amount of RFP protein was observed for only 3 days, whereas when the cells were treated with auto_shRFP@LS, the decrease in the amount of RFP protein was observed for the entire period of 10 days (FIG. 5B). These results demonstrate that the intensity of decrease in the amount of RFP protein was more effective when the cells were treated with auto_shRFP@LS.

Furthermore, as still another method for confirming the silencing of RFP gene by auto_shRFP@LS nanoparticles, the inhibition of fluorescent signal of RFP protein expressed in cells was observed by flow cytometry. B16F10/RFP cancer cells were treated with auto_shRFP@LS (final concentration: 15 μg/mL), and the intensity of RFP protein was observed after 0, 3, 4, 7, and 10 days.

As a result, when the cells were treated with siRFP@LS (final concentration: 50 nM siRFP) (a control group), the decrease of RFP fluorescent signal was observed for only 4 days, whereas when the cells were treated with auto_shRFP@LS, the RFP fluorescent signal was observed for at least 10 days. Additionally, when the cells were treated with auto(−)_shRFP@LS where the self-amplification of T7 polymerase is omitted (another control group), the decrease of RFP fluorescent signal was observed for at least 7 days, but the intensity of the decrease was weaker compared to when the cells were treated with auto_shRFP@LS (FIG. 5C). These results also demonstrate that auto_shRFP@LS nanoparticles can effectively and continuously express shRFP, thus being capable of inhibiting the RFP gene expression for a long period of time.

FIGS. 5D and 5E show the comparison results between the existing nucleus-dependent shRNA expression system (pSuper_shRFP@LS) and the auto_shRFP@LS of the present invention, thereby showing the specific difference between them with regard to a gene inhibition effect and its duration. Specifically, B16F10/RFP cells were treated with auto_shRFP@LS and pSuper_shRFP@LS at the same concentration (15 μg/mL), and the amount of decrease of intracellular mRNA of RFP was measured using the qRT-PCR method (FIG. 5D).

As a result, when the cells were treated with pSuper_shRFP@LS, the decrease in the amount of RFP mRNA was observed for 3 days, whereas when the cells were treated with auto_shRFP@LS, the decrease in the amount of RFP mRNA was observed for at least 7 days. Additionally, when the decrease of fluorescence intensity in the RFP fluorescent protein in cells by flow cytometry was measured, the cells treated with pSuper_shRFP@LS showed a decrease of fluorescence intensity only for 3 days, whereas the cells treated with auto_shRFP@LS showed an effective decrease of fluorescence intensity even on the 4th day. These results demonstrate that the limitation of short duration of inhibition raised in the existing nucleus-dependent shRNA expression system can be effectively resolved by the auto_shRNA@LS nanoparticles of the present invention.

### Example 7. Examination of Cancer Accumulation Property of Auto_shRNA@LS Nanoparticles in Mouse Xenograft Model

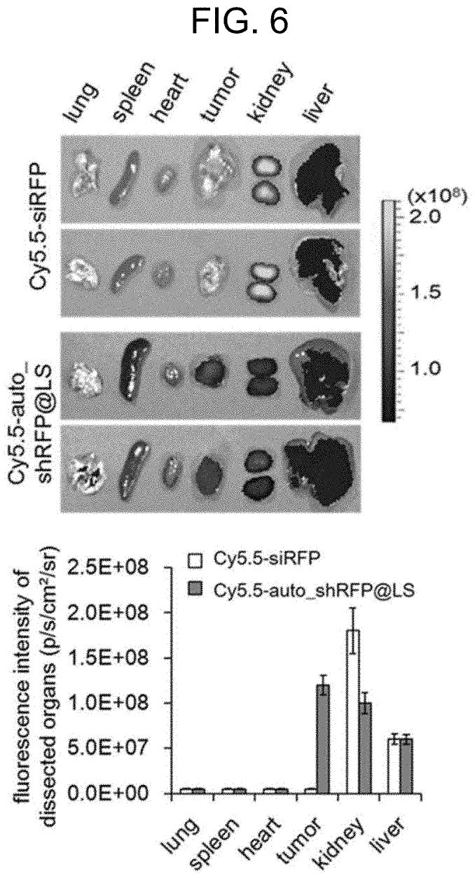

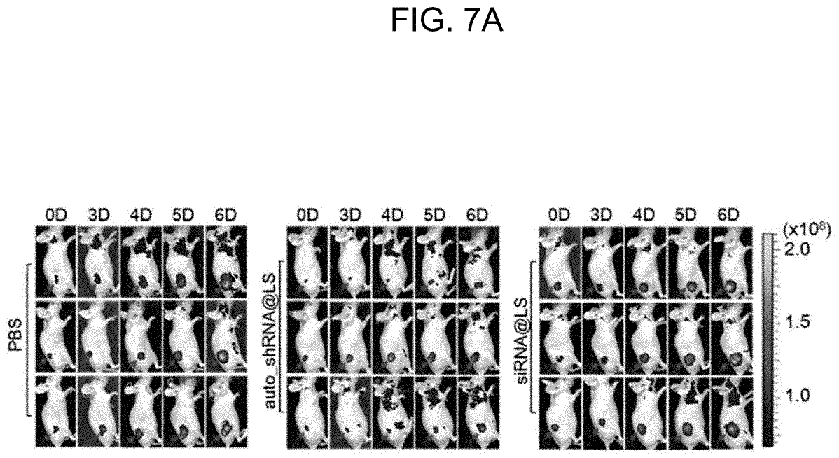

B16F10 cancer cells (1×10⁷) were inoculated into the left thigh of 5-week-old female BALB/c nude mice and allowed them to grow so that the volume of cancer tissue grew to 80 mm³. Fluorescently-labeled Cy5.5-auto_shRFP@ LS nanoparticles were intravenously injected into the caudal veins of the mice (50 μg), and major organs and cancer tissues were removed after 24 hours, and the accumulation level of fluorescently-labeled Cy5.5-auto_shRFP@LS nanoparticles was measured using the IVIS Spectrum (Caliper Life Science Inc., USA) (FIG. 6).

As a result, when the mice were intravenously injected with the fluorescently-labeled Cy5.5-siRFP (control group), the accumulation level was highest in the kidneys, whereas when the mice were intravenously injected with Cy5.5-auto_shRFP@ LS nanoparticles, the accumulation level was highest in cancer tissues. These results demonstrate that auto_shRFP@LS nanoparticles can be effectively and selectively accumulated in cancer tissues by the EPR effect.

### Example 8. Examination of Gene Silencing of Auto_shRNA@LS Nanoparticles in Mouse Xenograft Model

B16F10 cancer cells (1×10⁷) expressing RFP fluorescent protein were inoculated into the left thigh of 5-week-old female BALB/c nude mice and allowed them to grow so that the volume of cancer tissue grew to a size of 5 mm to 7 mm, and those mouse models where strong RFP fluorescent signals were observed were prepared. auto_shRFP@ LS nanoparticles (40 μg) were intravenously injected to the mice, and the changes in the fluorescence intensity of RFP were observed in the cancer tissue using the IVIS® Spectrum for a certain period of time (FIGS. 7A and 7B). As the control group, mice intravenously injected with siRFP@LS were prepared.

As a result, it was confirmed that the RFP fluorescence signal increased in proportion to the growth of cancer in PBS-injected mice with time. However, in the mice intravenously injected auto_shRFP@LS, only cancer tissue grew while the fluorescence intensity in the cancer tissue was inhibited. In the mice intravenously injected with siRFP@LS, it was observed that the fluorescence intensity was decreased for the first three days in the cancer tissue, but the fluorescence intensity on the 6th day was similar to that of PBS buffer-injected mice. Unlike existing siRNA delivery methods where intravenously injections are repeatedly administered at intervals of 2 to 3 days, the auto_shRFP@LS nanoparticles of the present invention, even by one intravenous injection, can effectively silence the target gene in the cancer tissue for more than 6 days.

After 6 days of the intravenous injection, cancer tissue was removed from the mice and the fluorescent signals were measured. As a result, strong fluorescence signals were observed in the cancer tissue of the control mouse, but relatively, significantly-reduced fluorescence signals were observed in the mice treated with auto_shRFP@LS nanoparticles (FIG. 7C). The removed cancer tissue was subjected to immunohistochemical staining using an anti-RFP antibody. As a result, it was observed that a large amount of RFP protein was stained in the cancer tissue of mice intravenously injected a PBS buffer or siRFP@LS, whereas a relatively lesser amount of RFP protein was stained in the cancer tissue of mice treated with auto_shRFP@LS nanoparticles (FIG. 7D). Additionally, the RFP mRNA remaining in the cancer tissue removed from the mice was quantified by qRT-PCR, and as a result, it was observed that a relatively large amount of RFP mRNA was reduced only in the cancer tissue of mice treated with auto_shRFP@LS nanoparticles (FIG. 7E).

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be included within the spirit and scope of the present invention as defined by the appended claims.

1. A method of sustained inhibition of gene expression comprising transferring a composition to a target tissue, which comprises an mRNA fragment of T7 RNA polymerase, plasmid DNA for self-amplification of the T7 RNA polymerase, and a DNA fragment encoding a gene expression inhibitor.

2. The method of claim 1, wherein the mRNA fragment consists of the nucleotide sequence of SEQ ID NO: 1.

3. The method of claim 1, wherein the mRNA fragment further comprises a 5′-cap structure.

4. The method of claim 1, wherein the mRNA fragment provides the first T7 RNA polymerase.

5. The method of claim 1, wherein the plasmid DNA consists of a T7 promoter, an internal ribosome entry site (IRES) domain, a gene encoding T7 RNA polymerase, a poly A tail, and a T7 termination sequence, which are operably linked.

6. The method of claim 1, wherein the plasmid DNA performs self-amplification of the T7 RNA polymerase by repetition of the expression loop of T7 RNA polymerase.

7. The method of claim 1, wherein the gene expression inhibitor is any one selected from the group consisting of siRNA, shRNA, microRNA, and an aptamer.

8. The method of claim 7, wherein the gene expression inhibitor is shRNA.

9. The method of claim 1, wherein the composition increases the expression of T7 RNA polymerase in the cytoplasm in a nucleus-independent manner and thereby expresses the gene expression inhibitor in a long-term sustained manner.

10. The method of claim 1, wherein the composition further comprises a transporter.

11. The method of claim 10, wherein the transporter is in the form of a liposome.

12. The method of claim 11, wherein the liposome consists of 1,2-dioleoyl-sn-glycero-3-phosphocholine (DOPC).

13. The method of claim 12, wherein the transporter is prepared by mixing a composition, which comprises an mRNA fragment of T7 RNA polymerase, plasmid DNA for self-amplification of the T7 RNA polymerase, and a DNA fragment encoding gene-expression inhibitor; and DOPC in a weight ratio of 1:5 (w/w) to 1:10 (w/w).

14. The method of claim 12, wherein the transporter is selectively accumulated in cancer tissue.

15. The method of claim 12, wherein the transporter delivers the gene expression inhibitor in a long-term sustained manner.

16. A method for preparing a transporter of a gene expression inhibitor, comprising:
(a) preparing each of an mRNA fragment of T7 RNA polymerase, plasmid DNA for self-amplification of the T7 RNA polymerase, and a DNA fragment encoding a gene expression inhibitor; and
(b) mixing the composition, which comprises the mRNA fragment of T7 RNA polymerase, plasmid DNA for self-amplification of the T7 RNA polymerase, and DNA fragment encoding gene-expression inhibitor; with DOPC.

17. The method of claim 16, further comprising removing free oligonucleotides by filtration, after step (b).

18. The method of claim 16, wherein the composition and DOPC are mixed in a weight ratio of 1:5 (w/w) to 1:10 (w/w).


SEQUENCE LISTING (13 sequences)

SEQ ID NO: 1 - DNA, 2655 bp, Bacteriophage T7
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataa

SEQ ID NO: 2 - DNA, 19 bp, Bacteriophage T7
taatacgactcactatagg

SEQ ID NO: 3 - DNA, 462 bp, Artificial Sequence
  pT7CFE1-NHis-IRES
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataa

SEQ ID NO: 4 - DNA, 2652 bp, Bacteriophage T7
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 5 - DNA, 48 bp, Bacteriophage T7
ctagcataaccccttggggcctctaaacgggtcttgaggggttttttg

SEQ ID NO: 6 - DNA, 66 bp, Artificial Sequence
  pT7/shRNA DNA template
taatacgactcactatagggagttcaagtccatctacattcaagagatgtagatggacttgaactc

SEQ ID NO: 7 - DNA, 64 bp, Artificial Sequence
  pT7/shRNA DNA template
gagttcaagtccatctacatctcttgaatgtagatggacttgaactctatagtgagtcgtatta

SEQ ID NO: 8 - DNA, 66 bp, Artificial Sequence
  pSuper_shRFP plasmid
ggatccccgagttcaagtccatctacattcaagagatgtagatggacttgaactctttttctcgag

SEQ ID NO: 9 - DNA, 66 bp, Artificial Sequence
  pSuper_shRFP plasmid
ctcgagaaaaagagttcaagtccatctacatctcttgaatgtagatggacttgaagtcggggatcc

SEQ ID NO: 10 - DNA, 19 bp, Artificial Sequence
  RFP mRNA-pF
gcgtgatgaacttcgagga

SEQ ID NO: 11 - DNA, 21 bp, Artificial Sequence
  RFP mRNA-pR
caatagtgatgacctggccgt

SEQ ID NO: 12 - DNA, 20 bp, Artificial Sequence
  beta-actin-pF
agagggaaatcgtgcgtgac

SEQ ID NO: 13 - DNA, 21 bp, Artificial Sequence
  beta-actin-pR
caatagtgatgacctggccgt